(19) United States
(12) Patent Application Publication
(10) Pub. No.: US 2007/0054349 A1
(43) Pub. Date: Mar. 8, 2007

(54) Biochip

(75) Inventor: Patrick Colin Hickey, Edinburgh (GB)

Correspondence Address: OSTRAGER CHONG FLAHERTY & BROITMAN PC, 250 PARK AVENUE, SUITE 825, NEW YORK, NY, 10177, US

(73) Assignee: LUX BIOTECHNOLOGY LIMITED, Einburgh (GB)

(21) Appl. No.: 10/573,223

(22) PCT Filed: Sep. 24, 2004
(86) PCT No.: PCT/GB04/04081

§ 371 Date: Mar. 23, 2006

(30) Foreign Application Priority Data
Sep. 24, 2003 (GB) 0323043.0

(51) Int. Cl. C12Q 1/18; C12M 1/34

(52) U.S. Cl. 435/32; 435/287.2; 257/E51.02

(57) ABSTRACT

A biochip comprising a substrate defining a plurality of fluid holding areas, the fluid holding areas separated by fluid separating elements for preventing mixing of fluids held in the fluid holding areas until the application of pressure to one or more of the fluids.

The present invention concerns a biochip, in particular a biochip adapted for screening a plurality of biomolecule-analyte interactions, and a method of fluid transfer for use with a biochip.

A biochip may be defined as a collection of miniature test sites onto which a number of biomolecules are attached with high density and in a defined microarray on a solid surface such as a silicon wafer. With a typical size of 1 cm², the biochip enables simultaneous tests to be conducted, facilitating high throughput of testing.

Many biomolecules are active only in solution or in the presence of a second molecule. However, often the activated form of the bio-molecule has a finite useful lifespan, thereby curtailing the shelf-life of any biochip containing it. In particular the need for water and nutrients to maintain viability has limited the use of micro-organisms (such as bacteria or fungi) in biochips.

It is an object of the present invention to address problems such as this.

According to the invention there is provided a biochip, comprising a substrate defining a plurality of fluid holding areas such as chambers, there being fluid separating means for preventing mixing of fluids held in said areas until the application of pressure to one or more said fluid. Thus, the present invention provides a biochip that is able to store a first biomolecule separately to a second molecule able to activate it, but wherein the first biomolecule and second molecule can be selectively mixed together to cause the first biomolecule to be activated when the biochip is required. This design of biochip has the advantage that the first biomolecule may be stored in an inactive form providing a longer shelf-life for the biochip.

The biochip may further include means for applying pressure to the one or more fluid. The means for applying pressure may comprise at least one expansible element, the arrangement being such that expansion of the or each expansible element results in the application of pressure to one or more fluid in a fluid holding area.

In a particularly advantageous embodiment, the expansible element is expansible upon application of light thereto at a suitable wavelength to cause heating of the expansible element.

It can thus be seen that the invention provides a method for fluid transfer which means that more complex designs of “lab-on-a chip” biochips can be constructed without the requirement for intricate electrical connections necessitated by prior art fluid transfer mechanics such as electrohydrodynamic pumps, electro-osmotic pumps, travelling wave pumps, piezoelectric pumps, magnetic pumps and peristaltic pumps (Biochip Technology, Cheng & Kricka 2001 Harwood Academic Publishers).

The separating means may be a membrane or film, preferably formed from a polymer (e.g. nitrocellulose, polyethylene, polypropylene) or an immiscible liquid (e.g. mineral oil, vegetable oil, paraffin etc) or a metal which is liquid above 5 degrees Celsius (e.g. mercury metal or the non-toxic alloy Galestan, containing gallium, indium and tin, described in U.S. Pat. No. 5,800,060 Speckbrock et al.).

Alternatively the separating means may be a metal which is solid at room temperature (e.g. gallium) but becomes liquid at raised temperature (30 Celcius), as a result of heating induced by a laser beam.

The first reactant may be a micro-organism present in an inactive form, for example as a spore. Mention may be made of fungal spores in this regard, but bacterial spores or other inactive forms of bacteria may also be used in the biochip. In this embodiment, the second reactant maybe water, or may be a mixture of water and nutrients (e.g. sugars, amino acids, and/or metal ions) required to stimulate activation/germination and growth of the micro-organism.

The upper surface of the biochip may contain a perforation (preferably 10-500 μm diameter) or porous membrane or filter to allow transfer of air in and out of the chip. This porous membrane may be filter paper (e.g. Whatman chromatography paper), a semi-permeable membrane (e.g. dialysis membrane, or a perforated film, preferably polyethylene). This perforation or membrane will allow for the displacement of air within chambers of channels of the biochip and in the case of biochips containing living organisms, will facilitate the transfer of oxygen and carbon dioxide required for metabolism. In the case where anaerobic organisms are contained within the chip, it may be sealed. Alternatively, a membrane can be included on top of the chambers as a means of injecting substances from outside. In this case, preferably the membrane is a self-sealing membrane made from silicone, latex or rubber similar to that contained on injection vials for dispensing drugs.

The lower surface of the biochip preferably comprises a transparent material (e.g. glass, polycarbonate, or polystyrene, but not limited to these materials). The transparent base layer permits microscopic examination of the samples in reactant chambers, and also allows transmission of laser energy to the microfluidic components.

Alternatively, the first reactant may be a protein or nucleic acid which requires the second reactant for activation. For example, certain enzymes require the presence of a co-factor or substrate (e.g. metal ions, ATP, ADP and for luciferase, they require a luciferin substrate e.g. coelenterazine) for activity and these combinations would be suitable for use in the present invention.

Alternatively in a three component reaction chamber, the first reactant may be a sample of cells, the second reactant may be a fluorescent dye or probe, and the third reactant may be a fixative e.g. paraformaldehyde. In this example, the living cells are first treated with a fluorescent dye or probe (e.g. Propidium Iodide, DAPI, FM4-64™, incubated for a period or alternatively immediately fixed with the fixative (reactant three).

Alternatively, in a four component reaction chamber, the first reactant may be a sample of cells, the second reactant may be growth medium, the third reactant may be a substrate, fluorescent dye or probe, and the fourth may be an unknown test substance.

Alternatively, five, six or more component systems may be incorporated in the biochip, resulting in complex multi-component laboratory processes to be carried out. The design of a multi-component biochip is simple due to the absence of electronic wiring, and thereby will reduce the cost, complexity and time taken for manufacture.

This site-specific injection provided by the invention is achieved using light (for example laser) stimulated fluid transfer/injection. A laser beam is directed, via an objective lens, or fibre optics or other optical mechanism, or directly from the laser source to a site on the chip composed of a light absorbing material which expands rapidly. This material may be a liquid, e.g. water, or an aqueous suspension of activated charcoal, colloidal suspension, glycerol, oil (e.g. mineral oil) gel (e.g. agarose) or polymer. Adjacent to this site is a chamber containing the fluid to be injected. Localised heating of the laser-irradiated area results in expansion of the material and forces the liquid into the chamber. Preferably the light absorbing material is separated from the reactant by an immiscible and inert fluid, thereby acting as a buffer to push the liquid within the microfluidic channel. The expanding material may be separated from the reactant by another liquid or gel that is inert and immiscible (e.g. when the expanding material is water containing a suspension of activated charcoal, it is desirable to be separated from reactant by an inert fluid e.g. mineral oil), providing physical separation from the heated material that may damage the reactant.

An alternative separating material may be a thin film that seals a channel, but is easily ruptured when the appropriate pressure is applied. The thin film may consist of nitrocellulose membrane, or polyethylene or other polymeric material. The pressure of fluid breaks the temporary seal of the separating material which prevents the liquid (e.g. reactant) flowing into chambers prematurely. As mentioned, an advantage of this method is that the biochip does not require electronic wiring, or external microinjection apparatus. The use of a laser to activate individual chambers means that highly accurate control can be achieved without perturbing the samples.

It is envisaged that the biochip may be mounted onto plastic cassettes that fit into the test chambers of commercially available luminometer or fluorometer equipment. Alternatively, the biochip may be imaged using a light microscope (e.g. laser scanning confocal microscope) or CCD camera device, either directly mounted on a CCD chip or viewed with the appropriate optics, e.g. a lens or fibre optic taper. Alternatively the biochip may be mounted on a device that supplies a light source e.g. a LED or solid state laser diode array.

As mentioned above, the mixing of the first and second reactants is achieved by displacement of a separating means through use of a laser. The accuracy of focus achievable with a laser beam enables predetermined chambers within the biochip to be selectively activated and this ability to select specific chambers for activation represents a significant advance in the art. Laser activation of biochips using a pulsed or scanning laser allows many operations to be controlled simultaneously. In addition, by varying the power of the laser, an accurate element of control is possible, enabling the volume and speed of injection to be regulated.

In one embodiment the first reactant is a fungal spore immobilised onto the chamber. The spores may be held in a matrix which is easily hydrated to achieve fast activation. The matrix may be an acrylamide based polymer or hydrogel or a filter paper.

Test substances may be added onto the biochip using array spotter or inkjet technology. The biochip is then sealed to retain moisture within the chambers, although, as mentioned previously, they may contain apertures or porous membranes to allow air transfer.

The biochip may be formed from any suitable base material typically a silicon wafer. Advantages of the silicon wafer are that they are transparent to infrared radiation, which in the case of an infrared laser used for heating, allows the laser light to be applied from the opposite side of the biochip. Disadvantages of silicon are it's inherent hydrophobic properties, although this can be altered by etching different surface textures, or application of another hydrophilic material to aid in water retention or adhesion of materials e.g. proteins/living cells. Other base materials which may be contemplated include silicon dioxide, indium tin oxide, alumina, glass, quartz, and metal (e.g. platinum, stainless steel and titanium). Moulded plastics (e.g. polypropylene, polyethylene) polymers (e.g. nitrocellulose) or ceramics may also be suitable.

Generally the base material is micro-machined to have the desired configuration of chambers and channels. Micro-machining may be carried out using techniques known in the art or in the related art of semi-conductor and electronics manufacture, for example, laser ablation, electro-deposition, vapour deposition, chemical etching, dry etching, photolithography and the like. In its simplest form the biochip may comprise a grid pattern of separate chambers etched onto a silicon wafer. In a more complicated form it will include channels, connections between separate chambers and arrays of different chambers.

An alternative to a silicon or glass substrate for the body of the biochip are the use of polymer materials. Suitable polymers include silicone polymers and thermoplastics such as polycarbonate, polypropylene and polymethylacrylate. The most efficient process of manufacturing such devices utilises negative or inverse replicas termed a “mould” or “replication master”. The replication master may be microfabricated from a hard and durable substrate such as silicon, glass or metal, using the techniques outlined above, and this is used to “print” multiple copies. Mass replication technologies used to print these polymeric biochips include hot embossing and injection moulding. Other methods used to directly microfabricate polymeric materials may utilise casting and laser micromachining.

In order to bond the biochip body to other materials (e.g. a transparent base, or multiple layers) may be achieved using lamination, adhesives, thermal bonding or laser welding.

The first and second reactants, and any other ingredients to be contained within the chamber, may be located onto all or any of the chambers on the pre-micro-machined base material. As the skilled worker will appreciate, known techniques such as ink-jet technology, array spotting or micro injection may be used for accurate placement of pre-determined aliquots of each ingredient/reactant.

To prevent adhesion of cells or other reactants to the channels of the biochip, a layer of non-stick material (e.g. Teflon®) may be applied.

Once the first reactant, the separating means and the second reactant have been located in the biochip, the biochip is sealed with a suitable outer layer. The outer layer should be strong enough to withstand damage and should also prevent leakage and evaporation. Mention may be made of nitrocellulose polypropylene as being suitable materials. A glass cover slip may be used allowing the entire biochip to be viewed using a microscope.

A preferred first reactant is fungal spores, in particular spores of filamentous fungi. Suitable fungi include Aspergillus sp. and Neurospora sp. A yeast such as Saccharomyces cerevisiae may also be used.

Optionally the fungi will have been bio-engineered to luminesce or fluoresce in the presence of a pre-selected analyte.

Optionally, the luminescence output varies in response to the presence or absence of the pre-selected analyte. Optionally the luminescent protein is a foreign protein and the filamentous fungus is genetically engineered to express that protein and to be luminescent, by introduction of the relevant gene as described for example in WO2004076685.

The gene for a luminescent protein may be obtained from firefly (Photinus pyralyis), crustaceans (Cypridina hilgendorfii), dinoflagellates (Pyrophorus noctilucus, Gonyaulax polyhedra), coral (Renilla reniformis) or naturally luminescent fungi (Panellus stipticus). Use of luminescent proteins of bacterial origin are also possible.

Preferred luminescent proteins include luciferase proteins, for example from Gaussia. Suitable genes expressing luminescent proteins are described in WO-A-99/49019.

Suitably the Gaussia luciferase is genetically engineered into Neurospora crassa, and optimised for mammalian codon usage. This mammalian gene can be successfully expressed in filamentous fungi.

Gaussia luciferase may be expressed in other species of filamentous fungi including Aspergillus nidulans, Aspergillus niger, Aspergillus fumigatus, Magnaporthe grisea and Sclerotinia sclerotiorum (a plant pathogen). Gaussia luciferase gene may be codon-optimised for codons preferred by filamentous fungi in order to increase light output. Other novel luminescent and fluorescent proteins (e.g. the calcium-sensitive Obelin photoprotein, and the Ptilosarcus green fluorescent protein, as described in U.S. Pat. No. 6,436,682) may also be expressed in filamentous fungi.

Alternatively the first reactant is a purified protein, (for example biotinylated Gaussia luciferase) and the second reactant is a substrate (e.g. coelenterazine). In this embodiment, the reaction of the purified protein with the substrate is initiated by laser stimulated transfer of the second reactant into the chamber containing the first reactant. Preferably the first reactant (biotinylated Gaussia luciferase) is covalently bound to the biochip, in this case through biotin-streptavidin conjugation.

The expression of the luminescent protein in living cells is desirably under the control of a gene promoter or enhancer sensitive to the presence of the pre-selected analyte to be assayed in the biochip.

Injection of the liquid into the biosensor chambers can be accomplished in different ways. To fill all chambers, the liquid is injected through channels which connect with all or a selected group of chambers on the array. The flow of liquid may be regulated by allowing it to flow through an absorbent material ensuring uniform distribution.

An alternative form of separating means comprises formation of a hydrophobic region directly on the channel or capillary that prevents movement of fluid along the capillary until it is propelled. The process of silanization may be used to alter the hydrophobicity of the substrate and prevent biological molecules from sticking to the surface.

The present invention will now be illustrated by way of example and with reference to the following figures in which:

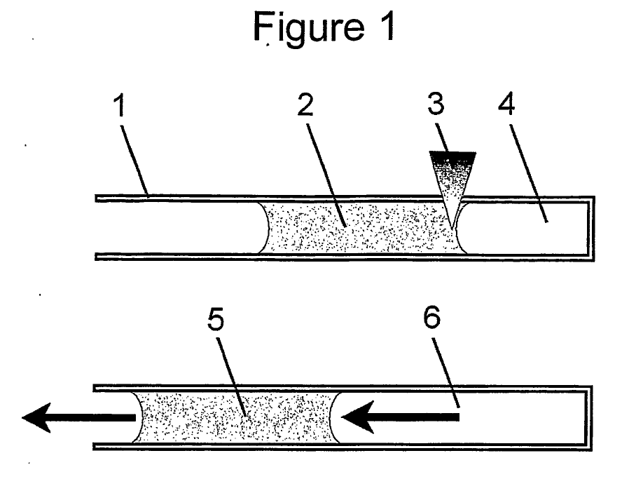

FIG. 1 is a schematic diagram showing the arrangement of a prototype capillary tube laser activated pump. During laser irradiation liquid is pushed along the tube.

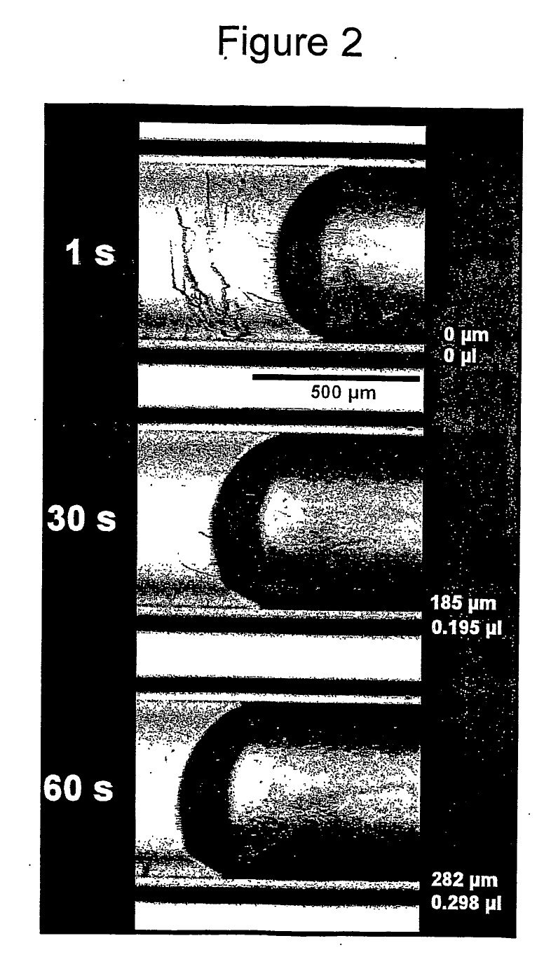

FIG. 2 shows images of a capillary tube laser activated pump at 1 second (1 s), 30 seconds (30 s) and 60 seconds (60 s) of irradiation with a 870 nm laser beam;

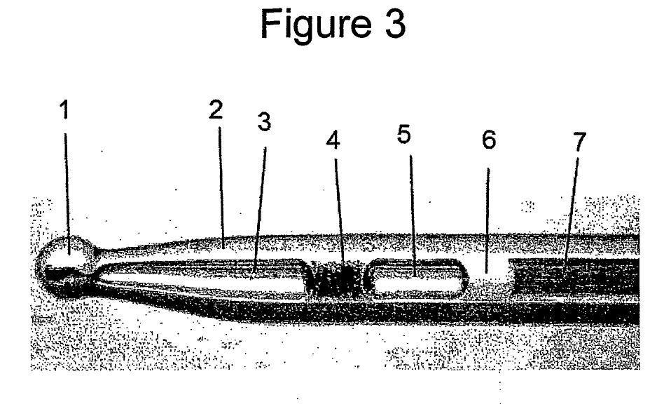

FIG. 3 shows a micro capillary loaded with three components;

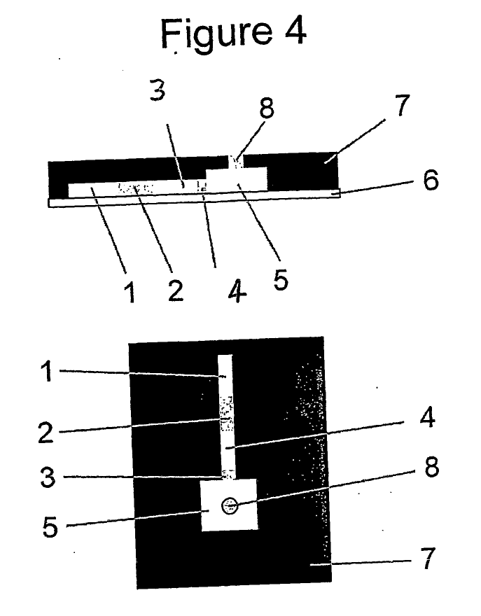

FIG. 4 shows an example layout for a biochip;

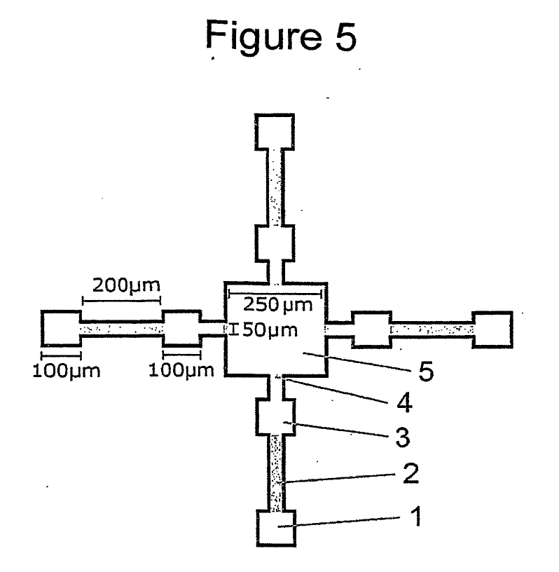

FIG. 5 shows an example of a multi-component biochip;

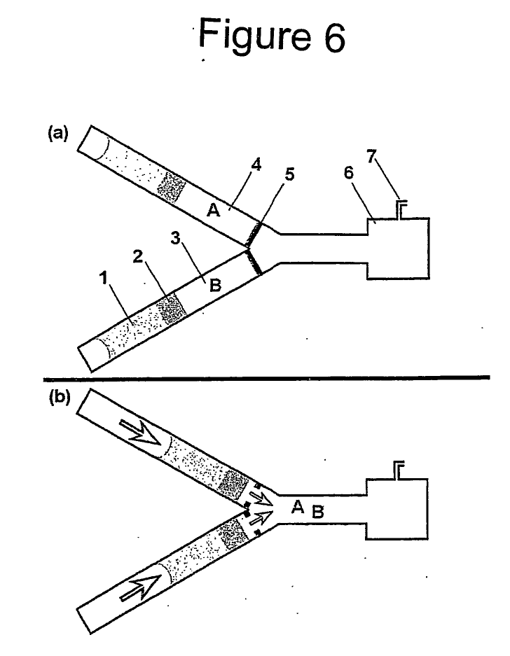

FIG. 6 shows a two component mixing system;

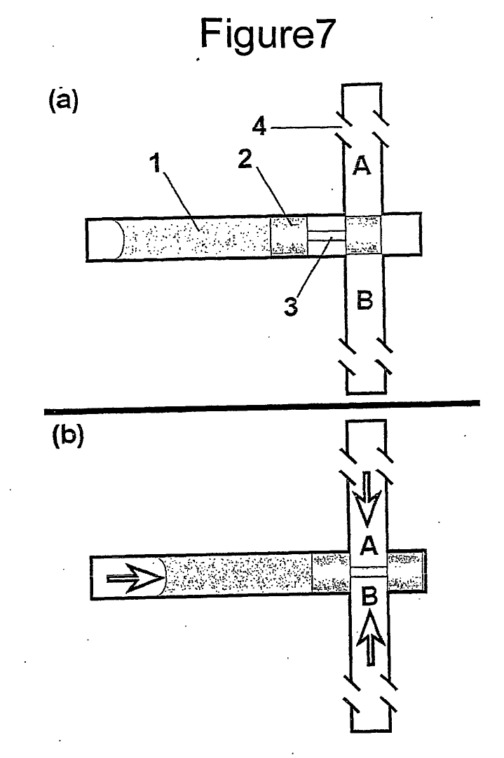

FIG. 7 shows a proposed piston-activated system;

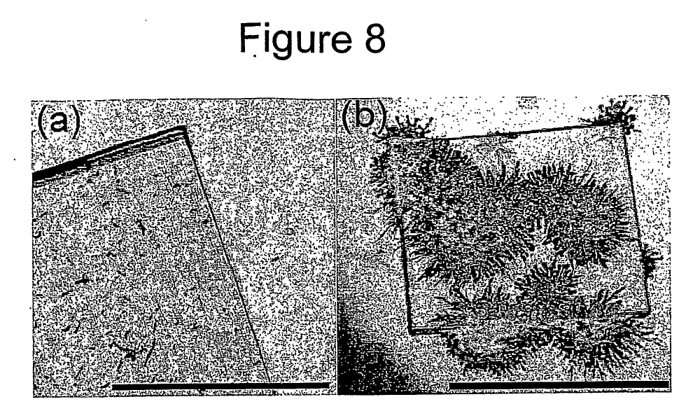

FIG. 8 shows a cellulose membrane coated with spores of Neurospora crassa;

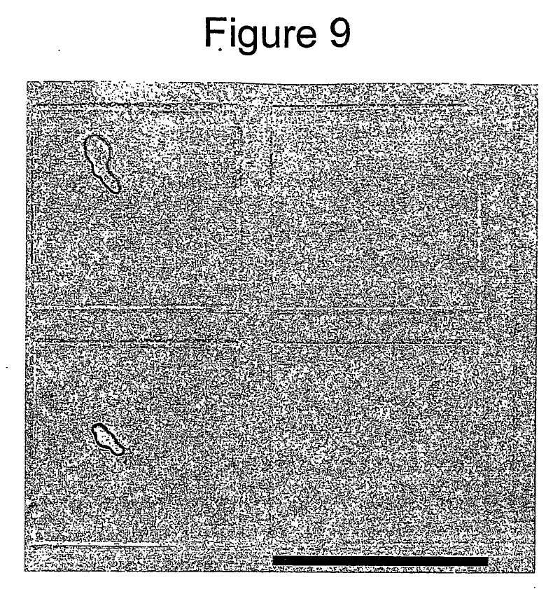

FIG. 9 shows (a) biochip populated with germinating spores of Neurospora crassa, and (b) Spores were hydrated for 2 hours and show growth. (Bar=100 μm); and

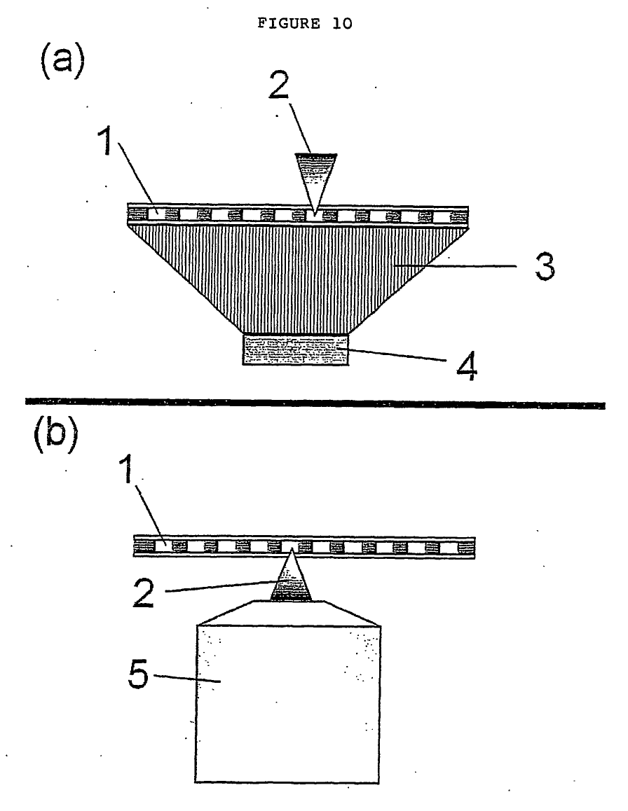

FIG. 10 shows the use of a biochip in an imaging system.

Referring to the drawings, and in particular FIG. 3, there is illustrated a glass capillary (2) with a sealed end (1), containing expansion material (4), a separating material (6) and reactant A (7). Air Bubbles (3,5) are present, due to the loading process.

Referring now to FIG. 4, the expansion material (1) (e.g. Water) is separated from Reactant A (4) by a separating material A, (2) (e.g. mineral oil). Reactant B (5) is located in a chamber and separated from reactant A by a separating material B (3) (mineral oil or a thin nitrocellulose membrane). The biochip body is tightly bonded to a transparent layer (6) (e.g. glass cover slip). Upon excitation of the expansion material (1) by laser radiation, it forces the separating material along the channel, breaking the separating material B, pushing reactant A into the Reactant B chamber. An opening (8), (e.g. pore, filter, re-sealable membrane or semi permeable membrane) on the upper side of the reactant B chamber (5) allows displaced air from reactant chamber B to escape and may also facilitate gas transfer for aerobic metabolism.

Referring now to FIG. 5, the expansion material (1) (e.g. Water) is separated from Reactant A (3) by a separating material A, (2) (e.g. mineral oil). Reactant B (5) is located in a chamber and separated from reactant A by a separating material B (4) (mineral oil or a thin nitrocellulose or plastic membrane). The biochip body is tightly bonded to a transparent layer (e.g. glass cover slip). Upon excitation of the expansion material (1) by laser radiation, it forces the separating material along the channel, breaking the separating material B, pushing reactant A into the Reactant B chamber. Multiple reactants may be added to the Reactant B chamber from each of the four injection systems shown on the diagram.

FIG. 6 shows a two component mixing system whereby reactant A (4) and reactant B (3) are contained in separate channels and the laser irradiation of expanding material (1) (water) causes the flow of the reactants together thereby mixing. A separating means (2) keeps the expanding material and reactant separate, and a second separating means (5) prevents premature flow of the reactants. This separating means (5) may be the form of a breakable membrane seal preferably nitrocellulose) or a hydrophobic region (e.g. silanised surface) which the hydrophobicity prevents capillary flow of the reactant. Reactants A and B are forced together and flow into the mixing chamber (6) and a vent (7) allows escape of displaced air or fluid following transfer of reactants A and B.

FIG. 7 shows a proposed piston-activated system whereby the laser irradiation of an expanding material (1) (water) pushes a piston (2) (preferably stainless steel, titanium, or plastic) across a channel (4), thereby allowing flow between contents A and B of the channel (4) which may or may not be under pressure. Preferably the piston is cylindrical dumbbell shape with a thinner connecting cylinder (3) in the middle which allows passage of fluid when it is engaged in the position shown in FIG. 7B. Alternatively, the piston is a cylinder with a central pore to allow flow when it is in the engaged position. FIG. 7b shows the actuated system in which the piston is engaged and allowing flow of the channel (4).

FIG. 8 (a) Cellulose membrane coated with spores of Neurospora crassa (Bar=0.5 mm). (b) Cellulose membrane after placing on agar for 24 hours results in germination of spores and formation of mycelial colonies (Bar=1 mm).

FIG. 9 (a) Biochip populated with germinating spores of Neurospora crassa. (b) Spores were hydrated for 2 hours and show growth. (Bar=100 μm).

FIG. 10 shows the use of a biochip in an imaging system. The biochip is designed to be imaged either using a contact imaging device such as a CCD chip coupled to an optical taper, or an inverted microscope. The laser beam can be applied to the biochip either directly, using the lens of a microscope, or applied from the opposite side.

### EXAMPLE 1

Laser irradiation of distilled water containing activated charcoal particles.

A liquid consisting of 10 mg activated charcoal per ml distilled water was drawn into a glass capillary tube of 1 mm outer diameter, 0.58 mm inner diameter. The activated charcoal was used since it possesses a dark colour which absorbs the maximum amount of light. One end of the capillary was sealed by melting the glass. The loaded capillary was placed in the stage of an inverted microscope and imaged using a X10 Plan Apo objective (NA=0.45). The multi-photon system consisted of a Bio-Rad Radiance 2100 with a Coherent Mira Ti-Sapphire laser tuned to 870 nm. The laser was used a full power and scanned for 50×2-second pulses. Upon irradiation, the laser energy caused the water to heat up, and boil. The boiling created water vapour, which pushed the liquid along the capillary tube. A schematic illustration of the experiments is illustrated in FIG. 1.

FIG. 2 shows images of the capillary tube at 1 s, 30 s and 60 s of laser irradiation. At 30 s, 0.195 μl of water has been pushed along the tube. After 60 s, 0.298 μl of water has been pushed along the tube. The irregular black lines with the water are moving particles of activated charcoal. The movement of the water clearly demonstrates that a laser can be used to cause a flow of liquid sufficient to facilitate mixing of the first and second reactants in a chamber of the biochip.

The mechanism that accomplishes this phenomenon is due to the following: Laser energy is absorbed by the charcoal/water suspension and results in localised heating, and subsequent boiling of the water. The expansion and vaporisation of the water pushes the liquid along the capillary.

### EXAMPLE 2

### Manufacture of a Biochip Containing Fungal Spores as a First Reactant

Spore Immobilisation

Cellulose membrane (cellophane) was cut into squares of 1.5 mm×1.5 mm. The membranes were then moistened with distilled water and sterilised in an autoclave. Spores of Neurospora crassa were harvested and suspended in distilled water. The spore solution was then added to the cellulose squares, coating them with spores (FIG. 8a). The cellulose squares were then placed in a Petri dish and dried in a laminar flow hood for 1-5 minutes. This rapid drying process ensures that the majority of spores remained dormant and thus allowing them survive storage. After 2 weeks storage in the dark at room temperature (20° C.) the spore-coated squares were placed on malt extract agar (2% malt extract; 2% agar) and incubated for 8 hours. After microscopic examination, it was noted that germination had occurred and mycelial colonies were developed (FIG. 8b).

Biochip

Nitrocellulose (pyroxylin) was dissolved in acetone and painted onto a silicon (approx 1.5 cm²) wafer with microfabricated squares of 100 μm×100 μm and 0.5 μm height. The nitrocellulose was allowed to dry for 20 minutes and then peeled off the silicon wafer. This process resulted in a “negative” imprint of the silicon wafer consisting of 100 μm square wells which were 0.5 μm deep. Spores were then deposited on the surface of the chip using a micropipette. Polylysine may be sprayed onto the chambers prior to introduction of spores. The polylysine acts as an adhesive to retain the fungal spores which may be accurately placed into each chamber using ink-jet technology, microinjection, array spotting or piezo-electric pump. Between 1 and 100 spores may be located per chamber of this design of biochip. The biochip was dried in a laminar flow hood at 25° C. The drying process was complete within 1-5 minutes thus ensuring that the spores remained dormant. Alternatively spores may be applied in an aqueous solution and dried using a freeze drier. This method ensures rapid drying and keeps the samples cold thus preventing degradation. For activation, the entire chip was then hydrated with 20 μl of distilled water. The chip was inverted and placed onto a coverslip (sandwiching the spores between the cellulose and glass). After 2 hours the sample was examined on a microscope and germination had occurred (FIG. 9). Spores were subsequently observed over a period of 4 hours, and exhibited normal growth.

Several biochip layers may be combined, each may contain growth media and substrates (e.g. coelenterazine) or fluorescent probes (e.g. propidium iodide, FM4-64). When use of the biochip is required, separating layers may be perforated by focusing a laser beam onto them. The spores will be activated following between 1 to 24 hours incubation at ambient temperature and the biochip will be ready for use. The biochip can be stored for several months without deterioration.

I claim:

1. A biochip, comprising a substrate defining a plurality of fluid holding areas, there being fluid separating means for preventing mixing of fluids held in said areas until the application of pressure to one or more said fluid, characterised in that the biochip comprises at least a first substance in a first fluid holding area, said first substance being in a substantially inactive or dormant condition, and a second molecule capable of activating the first substance in a second fluid holding area, the first and second fluid holding areas being separated by said fluid separating means.

2. A biochip according to claim 1, further including means for applying pressure to the one or more fluid.

3. A biochip according to claim 2, the means for applying pressure comprising at least one expansible element, the arrangement being such that expansion of the or each expansible element results in the application of pressure to the one or more fluid.

4. A biochip according to claim 3, wherein the expansible element is expansible upon application of light thereto at a suitable wavelength to cause heating of the expansible element.

5. A biochip according to claim 1, wherein the or each separating means comprises a frangible membrane or film.

6. A biochip according to claim 5, the membrane or film comprising a polymer.

7. A biochip according to claim 6, the polymer comprising nitrocellulose, polyethylene, or polypropylene.

8. A biochip according to claim 1, wherein the or each separating means comprises a fluid.

9. A biochip according to claim 8, wherein the fluid comprises mineral oil, vegetable oil, or paraffin.

10. A biochip according to claim 8, wherein the fluid comprises a metal which is liquid at room temperature.

11. A biochip according to claim 10, wherein the metal comprises mercury or Galestan.

12. A biochip according to claim 3, wherein the expansible element is a liquid.

13. A biochip according to claim 3, wherein the expansible element comprises an aqueous suspension of activated charcoal, a colloidal suspension, glycerol, oil, a gel or a polymer.

14. A biochip according to claim 1, further including a micro-organism in a first of said fluid holding areas, the micro-organism being in a substantially inactive or dormant condition, and a fluid in a second of said fluid holding areas in fluid communication with the first of said fluid holding areas and separated therefrom by a said separating means, the fluid being adapted to reactivate the micro-organism.

15. A biochip according to claim 14, wherein the micro-organism is a bacterium.

16. A biochip according to claim 14, wherein the micro-organism is a fungus.

17. A biochip according to claim 16, wherein the fungus has been bio-engineered to luminesce or fluoresce in the presence of a pre-selected analyte, such that the luminescence or fluorescence output varies in response to the presence or absence of the analyte, the fluid in the second said area comprising the analyte.

18. A biochip according to claim 14, wherein the reactivating fluid comprises or includes water.

19. A biochip according to claim 14, wherein the reactivating fluid comprises or includes a mixture of water and nutrients required to stimulate activation/germination and growth of the micro-organism.

20. A biochip according to claim 14, wherein the micro-organism is disposed in a hydratable matrix.

21. A biochip according to claim 20, the matrix comprising an acrylamide based polymer or hydrogel, or a filter paper.

22. A biochip according to claim 1, further including a protein or nucleic acid in one of said fluid holding areas, said protein or nucleic acid being in a form requiring activation.

23. A biochip according to claim 22, wherein the protein is an enzyme requiring the presence of a co-factor or substrate for activity.

24. A biochip according to claim 1, further comprising a cover disposed at its upper (in use) surface, the cover comprising one or more perforation.

25. A biochip according to claim 24, the cover comprising filter paper.

26. A biochip according to claim 24, the cover comprising a dialysis membrane, or a perforated film.

27. A biochip according to claim 24, the cover comprising a self-sealing membrane comprising silicone, latex or rubber.

28. A biochip according to claim 1, including a lower (in use) surface comprising of a transparent material.

29. A biochip according to claim 28, the lower (in use) surface comprising a glass, polycarbonate or polystyrene.

30. A biochip according to claim 1, wherein the substrate comprises silicon.

31. A biochip according to claim 1, comprising three fluid holding areas, a first containing a sample of cells, a second containing a fluorescent dye or probe, and a third containing a fixative, the areas being in fluid communication and separated from one another by separating elements.

32. A biochip according to claim 1, comprising four fluid holding areas, a first containing a sample of cells, a second containing a growth medium, a third containing a substrate, fluorescent dye or probe, and a fourth containing an unknown test substance, the areas being in fluid communication and separated from one another by separating elements.

33. A method of fluid transfer in a biochip according to claim 1, comprising the application of light to a portion of the biochip.

34. A method according to claim 33, wherein the light is laser light.

35. A method according to claim 33, wherein the light is applied to an expansible element, the light being adapted to cause heating of the expansible element which in turn causes displacement of the liquid.

36. A method according to claim 33, carried out on a biochip as claimed in claim 1.

37. A method according to claim 36, including the step of selectively activating fluid holding areas to achieve mixing of fluids.

38. A method according to claim 36, including the step of varying the power of the laser to regulate the volume and/or velocity of fluid transfer.
